(19) United States
(12) Patent Application Publication
(10) Pub. No.: US 2005/0152939 A1
(43) Pub. Date: Jul. 14, 2005

(54) Delivery technology

(76) Inventors: Craig Robert Bunt, Hamilton (NZ); Colin Roger Ogle, Hamilton (NZ); Michael John Rathbone, Hamilton (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/994,211

(22) Filed: Nov. 19, 2004

Related U.S. Application Data
(63) Continuation of application No. PCT/NZ03/00099, filed on May 22, 2003.

(30) Foreign Application Priority Data
May 22, 2002 (NZ) 519112

(51) Int. Cl. A61L 15/16

(52) U.S. Cl. 424/423; 604/500

(57) ABSTRACT

The present invention relates to a device for administration to the mammary gland cavity of a subject including at least one active agent; and, at least one carrier material; and, characterized in that the environment into which the device is inserted into activates dispersion of the active agent. In further embodiments, a muco-adhesive material is used as a retainer material for the carrier material/active agent mixture. The aim of the device is to deliver active agent or agents to a subject in the form of a suppository that, on administration to the subject, disperses into the subject environment. The device is particularly useful for delivery of an active agent to the mammary gland of a lactating cow.

### CROSS-REFERENCE TO RELATED APPLICATIONS

This application is a continuation of and claims priority under 35 U.S.C. § 120 to PCT Application No. PCT/NZ03/00099, filed on May 22, 2003 and published in English on Nov. 27, 2003 as WO 03/097109, entitled IMPROVEMENTS IN DELIVERY TECHNOLOGY; which application claimed priority to New Zealand Application No. 519112, which was filed on May 22, 2002; each of which applications is incorporated herein by reference in its entirety.

### BACKGROUND OF THE INVENTION

1. Field of the Invention

The invention relates to an improved delivery technology.

More specifically, the invention relates to a delivery device for administration to the mammary gland of a subject.

2. Description of the Related Art

For the purposes of this specification, the invention shall be disclosed in terms of administration to a cow however this is not limiting. It will be appreciated by someone skilled in the art that the delivery device can be administered to other animals besides cows.

The question of delivering an active agent to a subject has been considered at length in prior art. Indeed, there are many different known methods of delivering an active agent to a subject including sprays, infusions, injections, suppositories, pessaries, tablets and capsules.

Where the active agent must be delivered directly to a body cavity, the mode of administration is usually a suppository type device that is physically inserted into the cavity. These devices are useful for providing a dose of an agent to a subject as they can reach parts of the subject's body that other methods of administration cannot reach.

This method also has the disadvantage of not holding the deposit in place other than by normal bodily means. In worst cases the active agent leaks from the cavity thus not delivering the agent to the desired area for treatment.

Physical teat plugs in particular have been considered in previous embodiments to help retain the agent in the mammary gland of cows. One problem with this is that the plugs must be physically removed once treatment is complete. Where a large number of animals are being treated, this is a time consuming and labor intensive task.

In addition, the plugs are not always made of natural and/or biologically acceptable and/or physiologically acceptable materials. Furthermore, the plugs tend to fall from the teat thus wasting the active agent. A further problem is that plugs which fall off litter farm paddocks and can potentially become caught in and damage machinery.

A further problem with such devices is that the viability of the active agent can be reduced by a number of mechanisms prior to when the agent is delivered at the site to be treated. In worst cases the active agents breakdown entirely before insertion into the subject. For example air exposure and/or heat exposure during storage can lower the activity of unstable agents. Finally, agent release can be haphazard and not always controlled due to device constraints as well as the already described problems with retaining the device (and active agent).

It is therefore desirable to have a device that overcomes the above problems of securing the device, physical waste (of agent and device) and ‘bio-friendliness’ of the device.

In intra-mammary treatments, traditional methods have tended to utilize infusion methods for administration of the agent rather than a suppository device. A main reason for this usage is that the suppositories available are not of the appropriate shape and/or size for intra-mammary applications. The infusion method has the advantage of relative ease of formulation and administration.

Infusion methods do however have several disadvantages. Hygiene requirements mean that each syringe and/or cannula must be replaced for each teat. This is both time consuming and costly. Also, as the infusion formulation is typically viscous, the formulation may require warming to reduce the viscosity, thus allowing administration of the infusion. In worst case situations, active agent seeps out of the infusion site.

A further problem with infusion formulations is that they often require careful treatment such as refrigeration before administration to ensure the active remains stable.

An alternative to intramammary infusions includes dilators coated with a dextrin film containing salicylic acid is described in U.S. Pat. No. 2,832,343 (Mose). However the dilators must remain within the teat canal and be periodically replaced during the treatment period.

From the above discussion it can be seen that it is desirable to have an alternative to infusion methods for administering an active agent to a subject, particularly for intramammary applications.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### SUMMARY OF THE INVENTION

According to one aspect of the present invention there is provided a device for administration to the mammary gland of a subject including:
at least one active agent; and,
at least one carrier material; and,
characterized in that the environment into which the device is inserted into triggers dispersion of the active agent.

Preferably, the carrier material is also characterized in that the environment into which the device is inserted into triggers dispersion of the carrier material.

The carrier material and active agent combination is preferably adapted to fit the cavity. Most preferably, the carrier material is in an elongated shape to aid insertion into the cavity.

The present invention describes a useful device that has been found by the Applicant to be effective in delivering a dose of active agent to the mammary gland of a subject whereby the agent is kept stable before application and, on application, the device breaks apart or disintegrates to release the active agent. The elongated shape in particular, enables simple administration of the device to the mammary gland cavity.

Depending on the carrier material used, it will be appreciated by those skilled in the art that the rate of release of the active agent can also be regulated. By way of example, the active agent is located wholly within the carrier material and the dispersion of the carrier material (and subsequently, the active agent) is controlled by choice of carrier material and trigger mechanism.

In preferred embodiments, administration is by physical means including pushing the device into the cavity by hand. An alternative embodiment is to apply the device using an applicator device.

Preferably, the device is in a solid form capable of retaining its shape in order to allow insertion. It will be appreciated however that liquid active agents (and even gaseous agents) can also be utilized provided that the agent can be entrapped within the carrier material, thus the invention has a wide degree of flexibility in state of the active agent.

A further advantage of the device is that it is a single use complete device as opposed to an infusion method which requires replacement syringe, cannula and separate assembly. Also use of an infusion method requires skill in placement of the cannula point—such skill is not required in the present invention where the device need only be physically inserted into the cavity.

Preferably the cavity is a mammary gland. Most preferably the device is inserted into the lumen of the teat of the mammary gland.

The device is particularly suited to intra-mammary applications where the device is in contact with the walls of the cavity. In particular the device retains the active agent to be delivered in the cavity.

Preferably, the subject is an animal selected from species of the group consisting of bovine; cervine; porcine; ovine; bovidae.

In preferred embodiments, the subject is a lactating animal.

It is the understanding of the Applicant that the formulation can be used with any active agent. The agent can have biological, chemical, and/or physical activity but preferably the agent is biologically active.

Typical agents include those selected from the group consisting of: antibacterial agent; antifungal agent; anti-inflammatory agent; antiparasitic agent; anti-neoplastic agent; analgesic agent; anaesthetic agent; antipsychotic agent; vaccine; central nervous system agent; growth factor; hormone; antihistamine; osteoinductive agent; cardiovascular agent; anti-ulcer agent; bronchodilating agent; vasodilating agent; birth control agent; antihypertensive agent; anticoagulant; antispasmodic agent; fertility-enhancing agent; and combinations thereof.

It will be appreciated by those skilled in the art that, as the carrier material is preferably substantially inert with respect to the active agent, any type of active agent can be included.

In a further embodiment, a plurality of agents are used that act independently or in combination.

In preferred embodiments, the active agent is combined with the carrier material in a form selected from the group consisting of: a carrier material coating enclosing the active agent; an active agent coating on the outside of the carrier material; active agent randomly mixed through the carrier material; partial coating of carrier material on the active agent; and partial coating of active agent on the carrier material. Most preferably, the active agent is uniformly distributed throughout the carrier material.

Preferably, the carrier material or materials used are physiologically and pharmaceutically acceptable and are also substantially inert with respect to the active agent or agents. Examples of preferred carrier materials are those selected from the group consisting of: lactose; celluloses; cyclodextrins; starch; gelatin; dicalcium phosphate; calcium sulfate; kaolin; mannitol; sodium chloride; thermoplastics; stearates; and combinations thereof. Most preferably, the carrier material is selected from the group consisting of: lactose; magnesium stearate; and combinations thereof.

In the preferred embodiment, the environment into which the device is inserted triggers dispersion of the agent. Preferably this trigger for dispersion is selected from the group consisting of: moisture; pH; temperature; enzyme activity; air contact; other active agent activity; and combinations thereof. Most preferably, the trigger for dispersion is moisture content.

In preferred embodiments, the dispersion method is selected from the group consisting of: effervescence; liquid formation; gas formation; solid erosion; other known means for dispersion; and combinations thereof. Most preferably, the mechanism is dispersion in an effervescent manner like, for example a Berocca™ tablet placed in water.

In preferred embodiments, the rate of dispersion is adjusted by use of different carrier materials. More preferably, the rate of dispersion is within a period of 1 to 10 minutes.

Optionally, a retainer material is used to provide a barrier to retain the carrier material and active agent within the cavity. Preferably the retainer material falls out of the cavity or erodes away after the agent has been dispersed. For example, the retainer material drops out of the teat and degrades away naturally thereafter on the farm paddock. Preferably, the retainer material is made of physiologically and pharmaceutically acceptable materials. Most preferably the retainer material is a ‘muco-adhesive’ material.

For the purposes of this specification, ‘muco-adhesive’ refers to any polymer or material that when applied in a wet or dry form to a mucosal membrane, adheres in such a way as to slough off over a time period longer than that taken for dispersion of the active agent. Preferably muco-adhesive materials are selected from the group consisting of: polyethylene oxide; poly ethylene glycol; polyvinyl alcohol; polyvinyl pyrrolidine; poly acrylic acid; poly hydroxy ethyl methacrylate; hydroxypropyl cellulose; hydroxypropyl methylcellulose; hydroxyethyl methylcellulose; hydroxyethyl ethyl cellulose; hydroxyethyl cellulose; chitosan; and combinations thereof. Most preferably, the muco-adhesive material is hydroxypropyl methylcellulose or polyethylene oxide.

In preferred embodiments, the retainer material is applied to the carrier material active agent mixture at a point selected from the group consisting of: at least a portion of the carrier material/active agent mixture; dispersed within the carrier material/active agent mixture; enclosing the carrier material/active agent mixture; and combinations thereof. Most preferably the retainer material for elongated device applications is applied to: an end of the carrier material/active agent (‘layered’); as a complete coating to the exterior of the carrier material/active agent (‘a coated core’); as a partial coating to the exterior of the carrier material/active agent (‘a partially coated core’).

In further embodiments, the device can also contain further materials for administration to the subject. These materials are preferably physiologically and pharmaceutically acceptable, such as thickening agents, emulsifiers, stabilizing agents, glidants, lubricants and solubility enhancing agents.

According to a further aspect of the present invention there is provided a method of treatment of a subject by administration of a device substantially as described above.

According to a further aspect of the present invention there is provided the use of a device substantially as described above in the treatment of a subject.

It will be appreciated from the above description that there is provided a device that can be used quickly and relatively cheaply compared to injection methods.

It will be also appreciated by those skilled in the art that the above described device provides several advantages over traditional suppository administration devices including the use of the device for mammary gland applications.

It will also be appreciated that, as the carrier material and active agent mixture (and retainer material if present) can be shaped, administration is made easier.

In addition the device has a retainer material to hold the carrier material/active agent in place. This retainer material degrades naturally and does not require physical removal unlike traditional methods that both fall out of the cavity and pollute the environment into which they are released or remain within the subject until physical removal.

### BRIEF DESCRIPTION OF THE DRAWINGS

Further aspects of the present invention will become apparent from the ensuing description which is given by way of example only and with reference to the accompanying drawings in which:

FIG. 1 is a cross section side view of the teat as described in the preferred embodiment;

FIG. 2 is a further cross section side view of the teat as described in the preferred embodiment; and

FIG. 3 shows four preferred embodiments for the device.

### DETAILED DESCRIPTION OF THE PREFERRED EMBODIMENT

The invention will now be further described with reference to more detailed examples.

With reference to the attached drawings, the methodology and process is described below.

For the purposes of this example, the subject is a lactating cow wherein the device is administered to the mammary gland (2) of the cow. It will be appreciated by those skilled in the art that this description is given by way of example only and other subjects are also encompassed within the invention.

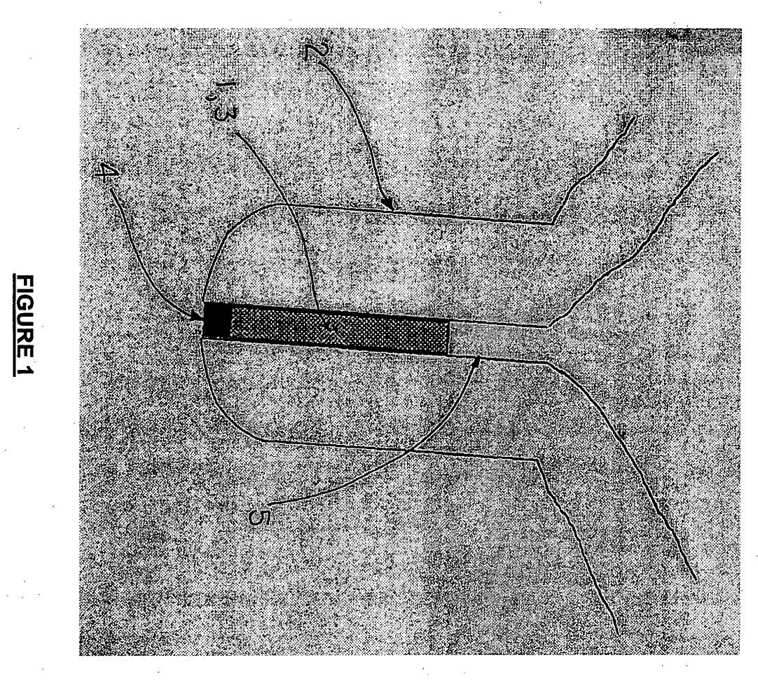

Referring to FIG. 1, an elongated shaped carrier material is used (3) containing a uniformly mixed biologically active agent (1) is inserted into the cow mammary gland (2). An end of the tablet is coated with a muco-adhesive material (the ‘retainer material’)(4). This material acts to retain the device (3) in the gland (2).

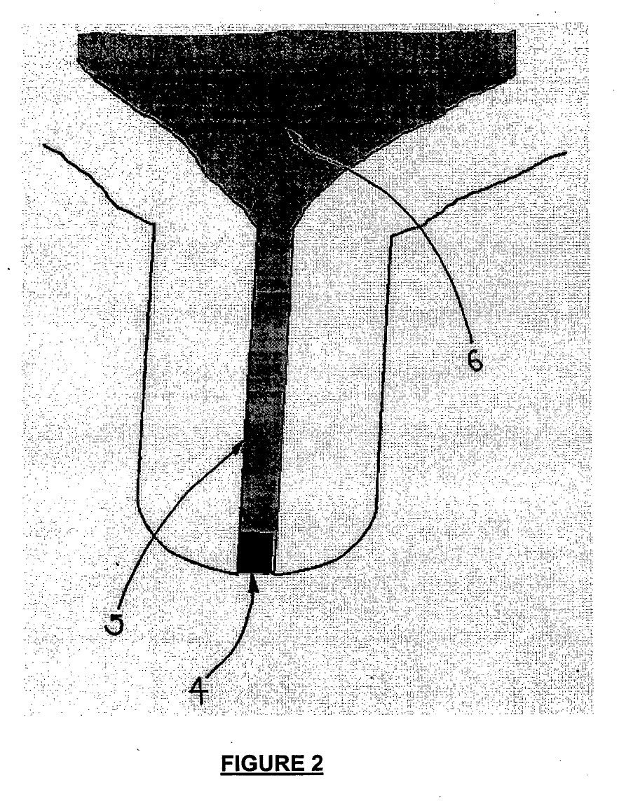

In the embodiment shown, the device (3) shows an effervescence option. The device (3) is in an elongated shape and is inserted into the lumen (5) of the teat. On contact with moisture in the lumen (5), the device (3) rapidly breaks down to that shown in FIG. 2 in an effervescent manner.

As shown in FIG. 2, the active agent is dispersed (6) in the mammary gland. The muco-adhesive portion (4), entering last, acts to retain the device in the lumen (5).

After a period of time following dispersion (6), the muco-adhesive portion (4) drops away from the lumen (5) of the teat.

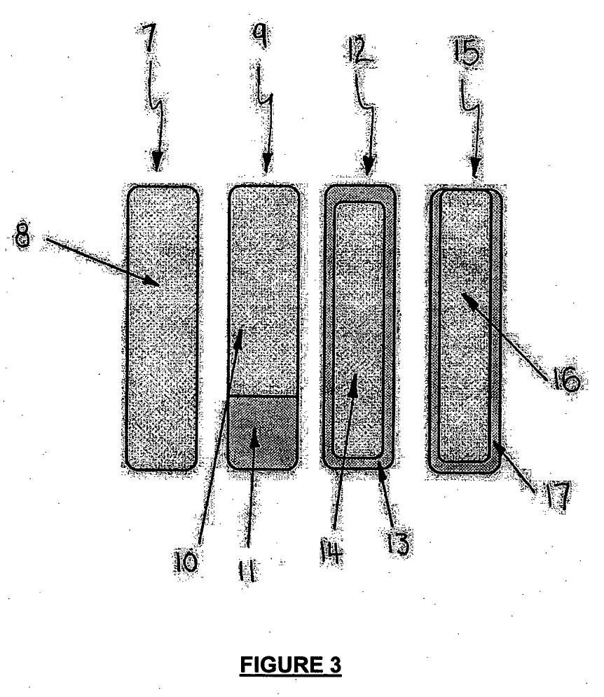

In the above example, one embodiment is shown for the device being a ‘layered’ option. This is further described in FIG. 3 and generally indicated by arrow (9) whereby the carrier material and active agent mixture (10) are evenly mixed together and a retainer material (11) is located at one end of the device. In this embodiment, the preferred carrier materials are a combination of lactose and magnesium stearate (1% wt). The retainer material is preferably polyethylene oxide.

Three other configurations are also shown, generally indicated by arrows (7), (12) and (15).

In the option indicated by arrow (7), a ‘monolithic’ example is shown whereby the device is made up of a carrier material core and active agent only (8). An example carrier material in this embodiment is a mixture of lactose and magnesium stearate (1% wt).

In the option indicated by arrow (12), a ‘coated core’ example is shown whereby the device is made up of a carrier material and active agent (14) completely enclosed within a retainer material coating (13). An example carrier material in this embodiment is a mixture of lactose and magnesium stearate (1% wt). The retainer material is preferably hydroxypropyl methylcellulose.

In the option indicated by arrow (15), a ‘partial coated core’ example is shown whereby the device is made up of a carrier material and active agent (16) partially enclosed within a retainer material coating (17). In this case, an opening exists at one end of the device however it will be appreciated that edges of the carrier material/active agent interior can be exposed on any edge or edges. An example carrier material in this embodiment is a mixture of lactose and magnesium stearate (1% wt). The retainer material is preferably hydroxypropyl methylcellulose.

It will be appreciated that a wide variety of different configurations are possible and only governed by the shape of the cavity and material characteristics.

Aspects of the present invention have been described by way of example only and it should be appreciated that modifications and additions may be made thereto without departing from the scope thereof as defined in the appended claims.

1. A device for administration to the mammary gland cavity of a subject, comprising:
at least one active agent; and,
at least one carrier material;
wherein the environment into which the device is inserted into triggers dispersion of the active agent.

2. The device of claim 1, wherein the carrier material is selected such that the environment into which the device is inserted into triggers dispersion of the carrier material.

3. The device of claim 1, wherein the carrier material and active agent combination is adapted to fit the cavity.

4. The device of claim 1, wherein the carrier material is in an elongated shape to aid insertion into the cavity.

5. The device of claim 1, wherein administration is by physical means including pushing the device into the cavity by hand,

6. The device of claim 1, wherein the device is administered using an applicator device.

7. The device of claim 1, wherein the device is in a solid form capable of retaining its shape in order to allow insertion.

8. The device of claim 1, wherein the device is inserted into the lumen of a teat of the mammary gland.

9. The device of claim 1, wherein the subject is an animal selected from species of the group consisting of: bovine, porcine, ovine, corvine, and bovidae.

10. The device of claim 1, claims wherein the subject is a lactating animal.

11. The device of claim 1, wherein the active agent is biologically active.

12. The device of claim 1, wherein the active agent is selected from the group consisting of: antibacterial agent, antifungal agent, anti-inflammatory agent, antiparasitic agent, anti-neoplastic agent, analgesic agent, anaesthetic agent, antipsychotic agent, vaccine, central nervous system agent, growth factor, hormone, antihistamine, osteoinductive agent, cardiovascular agent, anti-ulcer agent, bronchodilating agent, vasodilating agent, birth control agent, antihypertensive agent, anticoagulant, antispasmodic agent, fertility-enhancing agent, and combinations thereof.

13. The device of claim 1, wherein active agent is combined with the carrier material in a form selected from the group consisting of: a carrier material coating enclosing the active agent, an active agent coating on the outside of the carrier material, active agent randomly mixed through the carrier material, partial coating of carrier material on the active agent, and partial coating of active agent on the carrier material.

14. The device of claim 1, wherein the active agent is uniformly distributed throughout the carrier material.

15. The device of claim 1, wherein the carrier material is substantially inert with respect to the active agent.

16. The device of claim 1, wherein the carrier material used is physiologically and pharmaceutically acceptable.

17. The device of claim 1, wherein the carrier material is selected from the group consisting of: lactose, celluloses cyclodextrins, starch, gelatin, dicalcium. phosphate, calcium sulfate, kaolin, mannitol, sodium chloride, thermoplastics, stearates, and combinations thereof.

18. The device of claim 1, wherein the carrier material is selected from the group consisting of: lactose, magnesium stearate, and combinations thereof.

19. The device of claim 1, wherein the trigger for dispersion is selected from the group consisting of: moisture, pH, temperature, enzyme activity, air contact, other active agent activity, and combinations thereof.

20. The device of claim 1, wherein the dispersion method is selected from the group consisting of: effervescence, liquid formation, gas formation, solid erosion, other known means for dispersion, and combinations thereof.

21. The device of claim 1, wherein the rate of dispersion is adjusted by use of different carrier materials.

22. The device of claim 1, wherein the rate of dispersion is within a period of 1 to 10 minutes.

23. The device of claim 1, wherein the carrier material and active agent are retained within the cavity by a retainer material.

24. The device of claim 23, wherein the retainer material falls out of the cavity.

25. The device of claim 23, wherein the retainer material erodes away after the active agent has been dispersed.

26. The device of claim 23, wherein the retainer material is made of physiologically and pharmaceutically acceptable materials.

27. The device of claim 23, wherein the retainer material is a ‘muco-adhesive’ material characterized in that the material is a polymer or material that when applied in a wet or dry form to a mucosal membrane, adheres in such a way as to slough off over a time period longer than that taken for dispersion of the active agent.

28. The device of claim 27, wherein the muco-adhesive material is selected from the group consisting of: polyethylene oxide, poly ethylene glycol, polyvinyl alcohol, polyvinyl pyrrolidine, poly acrylic acid, poly hydroxy ethyl methacrylate, hydroxypropyl cellulose, hydroxypropyl methylcellulose, hydroxyethyl methylcellulose, hydroxyethyl ethyl cellulose, hydroxyethyl cellulose, chitosan, and combinations thereof.

29. The device of claim 23, wherein the retainer material is applied to the carrier material/active agent mixture at a point selected from the group consisting of: at least a portion of the carrier material/active agent mixture, dispersed within the carrier material/active agent mixture, enclosing the carrier material/active agent mixture, and combinations thereof.

30. The device of claims 23, wherein the carrier material is in an elongated shape to aid insertion into the cavity; and wherein the retainer material is applied to: an end of the carrier material/active agent (‘layered’), as a complete coating to the exterior of the carrier material/active agent (‘a coated core’), as a partial coating to the exterior of the carrier material/active agent (‘a partially coated core’).

31. The device of claim 1, comprising further physiologically and pharmaceutically acceptable materials for administration to the subject selected from the group consisting of: thickening agents, emulsifiers, stabilizing agents, glidants, lubricants, solubility enhancing agents, and combinations thereof 32. A method of treatment of a subject comprising administering a device of claim 1 to said subject.
